(19) United States
(12) Patent Application Publication
(10) Pub. No.: US 2018/0161264 A1
(43) Pub. Date: Jun. 14, 2018

(54) PHYSICALLY DRYING NAIL POLISH COMPOSITION, THE APPLICATION METHOD THEREOF, AND KIT INCLUDING SUCH A COMPOSITION

(71) Applicant: FlABILA, MAINTENON (FR)

(72) Inventors: Olivier NOUGUEREDE, HANCHES (FR); Anne-Lise LEGOUPY, PRUNAY LE GILLON (FR)

(21) Appl. No.: 15/578,844

(22) PCT Filed: Jun. 2, 2016
(86) PCT No.: PCT/FR2016/051315

§ 371 Date: Dec. 1, 2017

(30) Foreign Application Priority Data
Jun. 4, 2015 (FR) 1555081

(51) Int. Cl. A61K 8/81; A61K 8/85; A61K 8/73; A61K 8/37; A61Q 3/02

(52) U.S. Cl. CPC A61K 8/8147; A61K 8/85; A61K 8/731; A61K 8/37; A61K 2800/30; A61K 2800/81; A61K 2800/95; A61K 2800/884; A61Q 3/02

(57) ABSTRACT

The invention relates to a nail polish composition containing at least one film-forming agent, a plasticizer, and a solvent. Said composition is characterized in that it also includes one or more oligomer(s) having a molar mass of 1500 g/mol and/or a glass transition temperature Tg greater than 25° C. Said oligomer has dual acrylic or methacrylic bonds available for reacting with groups without containing any agent for cross-linking the oligomer, such as a photoinitiator. The method for applying the nail polish includes consecutively applying such a photoinitiator-free composition and a similar photoinitiator-containing composition followed by visible-light cross-linking. The nail polish has longer-lasting sheen and better hardness than solvent-based nail polishes and also comes on and off much more quickly than a gel-based nail polish.

The present invention relates to the field of nail polishes.

Nail polishes are currently classified in two main categories:

On the one hand, nail polishes are known that are based on nitrocellulose diluted with organic solvents, also containing a plasticizer, one or more resins and a thixotropic agent in the case of colored products. These polishes are easy to apply in one or more thin layers on the nails. Drying by evaporation of the solvent leads in a few minutes to the creation of a film that adheres to the nail and hardens. Removal is by application of cotton wool soaked in a nail polish remover solution (ethyl acetate, etc.), which dissolves the polish and removes it from the nail completely. These polishes do not keep their gloss long—it decreases after four days on average, and they have low wear resistance: depending on the person's activities, solvent-based polish applied on the nails must be renewed after a few days, a week at most.

To increase the strength and durability of the polish on the nail, polishes called “gel polishes” have also been proposed. Besides the film-forming polymer(s) based on cellulose, the basic composition contains UV-polymerizable monomers and/or oligomers of low molecular weight. After application of the gel polish on the nails, hardening of the polish therefore requires exposure of the latter for some minutes under an ultraviolet lamp to effect polymerization/crosslinking. Once crosslinked, these polymers give the polish greater strength and therefore longer durability on the nail. This very strong type of gel polish may then remain on the nail for at least two weeks without being damaged. However, nail growth means that application has to be renewed after this time. However, the improved strength of gel polishes relative to solvent-based polishes has a major drawback: these gel polishes are much more difficult to remove: removal of a solvent-based polish only requires simple rubbing with cotton wool soaked with solvent, but dissolution of a gel polish requires contact, or even direct immersion, of the nail with a nail polish remover (very often based on acetone) for at least ten minutes. This prolonged contact with a nail polish remover is also injurious to the nail. A longer application time, the need to use a UV lamp during application, and a long, tedious removal that is injurious to the nails discourage users from applying these gel polishes.

Moreover, the presence of crosslinking agents such as photoinitiators in the composition of the polish leads to two other drawbacks: on the one hand the presence of these photoinitiators means that, to prevent polymerization during storage, the polish must be packaged in opaque bottles, which makes it less attractive for the customer, who wants to see its color prior to purchase or use, and on the other hand the nails are brought into direct contact with the photoinitiators, which are generally chemicals that are sensitizing, allergenic and sometimes toxic.

There is therefore still a real need for a nail polish having the advantages both of the solvent-based polishes and of the gel polishes, without their respective drawbacks. The main aim of the present invention is to overcome the drawbacks of these two existing categories of nail polishes.

A first aim of the invention is therefore to propose a nail polish composition that is crosslinkable without exposure to a UV lamp.

Another aim of the invention is to propose a nail polish composition that has, after application on the nails, improved durability, notably better wear resistance and a longer lasting gloss relative to a solvent-based polish.

Another aim of the invention is to propose a nail polish composition having a make-up time that is identical to and make-up removal that is equivalent to that of a solvent-based polish.

Another aim of the invention is to propose a nail polish composition that does not contain sensitizing or toxic substances that come into contact with the nails.

For this purpose, the nail polish composition according to the invention, containing at least one film-forming agent, a plasticizer and a solvent, is characterized in that it also comprises one or more oligomer(s) of molecular weight above 1500 g/mol and/or whose glass transition temperature Tg is above 25° C., said oligomer possessing acrylic or methacrylic double bonds that are available for reaction with radicals, but do not contain a crosslinking agent of the oligomer, such as a photoinitiator.

Oligomers of this kind have the property of “physical” drying, i.e. after a few minutes, the polish applied on the nail is dry, and no longer displays tack, in contrast to the behavior of the gel polishes of the prior art. More particularly, the oligomer has a Persoz hardness of at least 60 s, preferably of at least 80 s, more preferably of at least 100 s, when it is applied on a glass plate at a thickness of 100 μm when wet (corresponding to 50 μm when dry), then dried for 24 h at 25° C. away from the light.

Advantageously, the molecular weight of the oligomer is greater than or equal to 2000 g/mol, preferably greater than or equal to 3000 g/mol.

This oligomer advantageously has a glass transition temperature Tg above 30° C., preferably above 35° C., more preferably above 40° C. This glass transition temperature Tg is measured on the crude oligomer before any crosslinking.

It was found that the oligomers of the (meth)acrylate type, therefore possessing acrylic or methacrylic double bonds available for reaction with radicals, in the absence of a photoinitiator and satisfying the above conditions, make it possible to obtain properties of improved gloss, better wear resistance, while having a make-up time that is identical to and make-up removal that is equivalent to that of a solvent-based polish.

The non-aromatic oligomers of the (meth)acrylate type are preferred, since oligomers containing aromatic rings have a tendency to turn yellow.

The oligomer of the (meth)acrylate type is preferably selected from: a (meth)acrylated poly(meth)acrylate, a (meth)acrylated polyester, a (meth)acrylated polyether, a (meth)acrylated urethane, a (meth)acrylated polyurethane or a (meth)acrylated epoxy or a mixture thereof.

Examples of these (meth)acrylates that can be used in the compositions according to the invention are the products:

Ebecryl 1200 from the company Allnex: acrylated poly(meth)acrylate,

Ebecryl 4654 from the company Allnex: acrylated polyurethane

Doublemer 347-1: from the company Double Bound: acrylated silane-modified polyacrylate

Lumicryl U721 from the company Estron: acrylated poly(meth)acrylate

Their properties are presented in Table 1 below.

TABLE 1

| | | Molar | | Persoz |
|---|---|---|---|---|
| | | mass | Tg | hardness |
| Oligomer | Nature | (g/mol) | (° C.) | (s) |
| Ebecryl 1200 | acrylated | >10 000 | 65 | 90 |
| | poly(meth)acrylate | | | |
| Ebecryl 4654 | acrylated | 15 000 | 92 | 210 |
| | polyurethane | | | |
| Doublemer 347-1 | acrylated silane- | >12 000 | 43 | 150 |
| | modified | | | |
| | polyacrylate | | | |
| Lumicryl U 721 | acrylated | >7000 | | |
| | poly(meth)acrylate | | | |

The values of the glass transition temperatures Tg of these oligomers (not crosslinked) were measured by differential scanning calorimetry using DSC equipment with power compensation, DSC Q2000 (TA instruments).

The operating conditions were as follows:
Mode: standard
Sealed aluminum sample-holder crucibles
Purge gas: U-grade nitrogen (50 ml/min)
Temperature ramp used:
   Isothermal at −90° C. for 5 minutes
   From −90 to 200° C. at 10° C./min
   Isothermal at 200° C. for 5 minutes
   From 200 to −90° C. at 20° C./min
   Isothermal at −90° C. for 5 minutes
   From −90 to 200° C. at 10° C./min
   Isothermal at 200° C. for 5 minutes

Advantageously, the nail polish composition according to the invention contains a concentration of said oligomer between 0.1 and 30 wt %, preferably between 0.5 and 20 wt %, more preferably between 1 and 15 wt % of the total weight of the nail polish composition.

Advantageously, the nail polish composition according to the invention has a content of solvent between 60% and 85%. This content of solvent makes it possible to have a sufficiently fluid composition, for easy application on the nails.

This type of composition leads to a shiny film on the nail, for a longer time than the conventional solvent-based polishes. Removal is easy, and does not require prolonged contact with nail polish remover.

A first type of nail polish composition according to the invention, called first composition or basic composition, does not contain a crosslinking agent of the oligomer, such as a photoinitiator, and it can therefore be applied directly on the nails, thus avoiding any problem of sensitization or allergy for the user.

A second type of nail polish composition according to the invention, usable as a finishing composition, also called “top-coat” composition or second composition, intended to be applied on the basic composition, additionally comprises a photoinitiator, preferably of the radical type, capable of initiating crosslinking of the oligomer or oligomers (at least) in daylight (i.e. without requiring the use of a UV lamp). Thus, the photoinitiator is not in direct contact with the nail.

The concentration of photoinitiator in said “top-coat” composition is, advantageously, between 0.1 and 10 wt %, preferably between 0.5 and 5 wt % of the total weight of the nail polish composition.

The present invention also relates to a method for applying polish on a nail, comprising the following successive steps:
i) applying a first composition A, as described above, on the nail, in the form of at least one film FA, composition A preferably containing a colored pigment and not containing a photoinitiator; followed by drying of said film FA;
ii) applying, on the surface of the film FA formed by composition A, a second composition B as described above, called top coat, in the form of a film FB, composition B containing a photoinitiator;
iii) initiating the crosslinking of composition B in visible light, with the crosslinking of the oligomers in composition B causing crosslinking of the oligomers present on the surface of film FA, inducing a strong bond between films FA and FB.

The presence of the oligomers of the first composition A allows said film FA to dry quickly, and thus allows subsequent application of the layer of the second composition B on the surface of the first film FA without delay.

The present invention also relates to a nail polish kit comprising:
a container CA, of the bottle type, containing a first nail polish composition A according to the invention, without photoinitiator,
a container CB, of the bottle type, containing a second nail polish composition B according to the invention containing a photoinitiator,
instructions for use.

Advantageously, container CA has walls that are at least partially transparent, so that the color of the colored polish of the first composition A is visible when the latter is stored in said container CA and contains a colored pigment. The user is thus able to find out the color of the colored polish directly.

Advantageously, container CB has walls that are opaque to visible light and to UV, able to prevent crosslinking of the polish of the second composition B containing the photoinitiator when the latter is stored in said container CB. The second composition B or top coat is generally colorless, or very slightly colored.

The kit according to the invention may additionally comprise a container containing a nail polish remover able to remove the layers of polish A and B from the nails, for example for make-up removal by means of cotton wool impregnated with said nail polish remover.

The present invention will now be described in more detail and illustrated by the non-limiting examples given below:

### EXAMPLES

Various nail polish compositions were tested, according to the present invention and compared with compositions of the prior art.

All the percentages stated below are percentages by weight.

Among the tests performed on the nail polishes obtained:
Gloss was measured (on a scale of 100) with a Minolta 268 glossmeter (angle of incidence 60°) for application (100 μm wet) on a card of the LENETA type.
The wet abrasion test consists of measuring the resistance to wear caused by a brush after to-and-fro movements of the latter on the surface of the film. For this, one or more films of 100 μm when wet are applied on an aluminum plate. After drying for 12 h at room temperature, the gloss of this system is measured. This is the value 0 to-and-fro movement. The system is then tested and the gloss is measured every 2000 to-and-fro movements. The higher the gloss, the more resistant the film.
Hardness was measured using a “Persoz” pendulum on the dry film formed by applying a layer of the composition, with thickness of 100 μm when wet, on a glass plate, and dried for 24 h at room temperature (25° C.); the value is expressed in seconds.
Adherence was measured by carrying out the Cross Hatch Test on the dry film formed by applying a layer of the composition, with thickness of 100 μm when wet, on a glass plate and dried overnight at 25° C. A score of 0 corresponds to no loss of adhesion. A score of 5 corresponds to total loss of adhesion.

### Comparative Example 1

### Conventional Solvent-Based Polish

This polish was obtained by applying two layers of colored composition and one layer of “top coat”. The respective compositions were as follows:

Colored Composition:

| | |
|---|---|
| Butyl acetate | 38 |
| Ethyl acetate | 20.7 |
| Nitrocellulose | 18 |
| Acetyl tributyl citrate | 9 |
| Adipic acid/neopentylglycol/trimellitic anhydride | 12 |
| copolymer | |
| Stearalkonium bentonite | 1.5 |
| Pigment Red 7 Lake | 0.8 |
| TOTAL | 100 |

Composition of Top Coat:

| | |
|---|---|
| Butyl acetate | 20 |
| Ethyl acetate | 46 |
| Cellulose acetate butyrate | 16 |
| Isopropyl alcohol | 10 |
| Adipic acid/neopentylglycol/trimellitic anhydride | 5 |
| copolymer | |
| Acetyl tributyl citrate | 3 |
| TOTAL | 100 |

This polish displays “physical drying”, i.e. we observe drying of the polish by evaporation of the solvent, and hardening in the air and in visible light after a few minutes.

### Comparative Example 2

### Gel Polish of the Prior Art

This gel polish, marketed under the name SHELLAC, from the company Creative Nail Design, comprises a set of three compositions: a basic composition, applied in a single layer directly on the nail and crosslinked under UV, two layers of a colored composition that are crosslinked under UV and a “top-coat” layer of a final composition covering the preceding layers, also crosslinked under UV. These three compositions contain a high proportion (between 40 and 60%) of an oligomer of the polyurethane acrylate type such as the Di Hema trimethylhexyl dicarbamate of molecular weight 470 g/mol, in the presence of other (meth)acrylates with molecular weight below 1000 g/mol (WO2011/011304). None of these compounds displays “physical drying” in the sense of the definition given in the introduction. However, these acrylated compounds polymerize rapidly under ultraviolet radiation to form a strong film on the nail. It is therefore essential to have a UV lamp for carrying out the successive polymerization/crosslinking of the various layers applied.

### Example 3

### Invention

This polish comprises a colored composition and a finishing composition (top coat), with the formulations presented below:

Colored Composition (Designated 1st Composition):

| | |
|---|---|
| Butyl acetate | 31 |
| Ethyl acetate | 31.2 |
| Nitrocellulose | 15 |
| Acetyl tributyl citrate | 6 |
| Adipic acid/neopentylglycol/trimellitic anhydride | 12 |
| copolymer | |
| Oligomer | 2.5 |
| Stearalkonium bentonite | 1.5 |
| Pigment Red 7 Lake | 0.8 |
| TOTAL | 100 |

Finishing Composition (Designated 2nd Composition):

| | |
|---|---|
| Butyl acetate | 19.4 |
| Ethyl acetate | 46 |
| Cellulose acetate butyrate | 15 |
| Isopropyl alcohol | 10 |
| Photoinitiator | 2 |
| Acetyl tributyl citrate | 2.6 |
| Oligomer | 5 |
| TOTAL | 100 |

In this example, the photoinitiator is ethyl (-2,4,6-trimethylbenzoyl)phenylphosphinate (TPO I) from the company Rahn, the oligomer is the product Doublemer 347-1 from the company Double Bound and the pigment is Red 7 Lake, Unipur Red LC 3071 from the company Sensient.

In contrast to the Shellac polish of comparative example 2, the 1st composition, that composition in direct contact with the nail, does not comprise a photoinitiator, but dries on the nail in a few minutes, allowing it to be covered with the finishing composition. The finishing composition contains a photoinitiator for accelerating and effecting complete polymerization/crosslinking of the oligomer in visible light, without requiring the use of a UV lamp.

Gloss tests were performed on this polish, as well as the polishes of comparative examples 1 and 2. In the case of example 2, the comparative polish was passed under a 36 W UV lamp after application of the basic composition (10 seconds), the first colored layer (2 minutes), the second colored layer (2 minutes) and the “top coat” layer (4 minutes). The whole was then defatted with isopropanol as indicated in the manufacturer's instructions.

Gloss at t₀

Example 1 (comp.)=82 UB

Example 2 (comp.)=85 UB

Example 3=88 UB

The results for the percentage loss of gloss as a function of the number of passes in the wet abrasion test are presented in Table 2 below.

TABLE 2

| | | Polish 1 | Polish 2 | Polish 3 |
|---|---|---|---|---|
| | | (Comp.) | (Comp.) | (invention) |
| 0 | pass | 100 | 100 | 100 |
| 2000 | passes | 99.1 | 100 | 100 |
| 4000 | passes | 98.8 | 99.6 | 99.5 |
| 6000 | passes | 97.6 | 99.3 | 99.3 |
| 8000 | passes | 96.3 | 99 | 99.1 |
| 10000 | passes | 96.3 | 98.7 | 98.8 |

The loss of gloss of the polish according to the invention is minimal, of the same order of magnitude as that of the gel polish.

The make-up and make-up removal times were also noted, and are presented in Table 3 below.

TABLE 3

| Polish | Make-up time | | Make-up removal time |
|---|---|---|---|
| Ex. 1 (Comp.) | 5 | minutes | 5 minutes with nail polish remover |
| | | | based on ethyl acetate |
| Ex. 2 (Comp.) | 25 | minutes | 30 minutes with nail polish remover |
| | | | based on acetone |
| Ex. 3 | 5 | minutes | 5 minutes with nail polish remover |
| | | | based on ethyl acetate |

It can be seen that the times for make-up and make-up removal of the polish according to the invention are equivalent to those of a conventional solvent-based polish, and much lower than those of the gel polish, without requiring the use of an acetone-based solvent.

A test was performed on a panel of 20 people. A first group tested polish 1, a second group polish 2 and a third group polish 3.

These panellists gave their opinion on the durability of the gloss of the system applied. The averages of the replies are presented in Table 4 below.

TABLE 4

| Polish | Durability of the gloss | |
|---|---|---|
| Polish 1 (Comp.) | 4 | days |
| Polish 2 (Comp.) | 10 | days |
| Polish 3 | 8 | days |

It is noted that the durability of the gloss is doubled relative to the conventional solvent-based polish.

Measurements of hardness (Persoz) were also performed on the polishes of examples 1, 2 and 3 after drying:
on the base polish (colored polish formed by applying two layers of 100 μm when wet)
on the preceding base polish, on which a “top coat” layer of 100 μm when wet was applied.
   The results are presented in Table 5 below.

TABLE 5

| | |
|---|---|
| | 2 layers of colored |
| 2 layers of colored | polish + 1 layer of |
| polish | top coat |

| | | | | |
|---|---|---|---|---|
| Ex. 1 (comp.) | 242 | s | 226 | s |
| Ex. 2 (comp.) | 36 | s | 25 | s |
| Ex. 3 (invention) | 267 | s | 251 | s |

Higher hardness is noted for the polish according to the invention before and after application of the finishing layer (top coat) even in the absence of photoinitiator for the layers of colored polish.

### Example 4

### Proportions of Oligomer and Photoinitiator

In this example, different percentages of oligomer and photoinitiator were used in the finishing composition (top coat) applied on the same “colored” polish as in example 3. The three additional finishing compositions are presented in Table 6.

TABLE 6

| | Polish 3 | | | |
|---|---|---|---|---|
| | of ex. 3 | Polish 4 | Polish 5 | Polish 6 |
| Butyl acetate | 19.4 | 20.4 | 17.4 | 19.4 |
| Ethyl acetate | 46 | 48 | 43 | 44 |
| Cellulose acetate | 15 | 15 | 15 | 15 |
| butyrate | | | | |
| Isopropyl alcohol | 10 | 10 | 10 | 10 |
| Photoinitiator | 2 | 2 | 2 | 4 |
| Acetyl tributyl | 2.6 | 2.6 | 2.6 | 2.6 |
| citrate | | | | |
| Oligomer | 5 | 2 | 10 | 5 |
| TOTAL | 100 | 100 | 100 | 100 |

The photoinitiator is ethyl (2,4,6-trimethylbenzoyl)phenylphosphinate (TPO I) and the oligomer is the product Doublemer 347-1 from the company Double Bound.

Similarly to example 3, measurements of gloss, loss of gloss in a wet abrasion test (Table 7), make-up and make-up removal times (Table 8) were performed, and are presented below.

Gloss at t₀

Polish 3=88 UB

Polish 4=86 UB

Polish 5=89 UB

Polish 6=87 UB

The results for the percentage loss of gloss as a function of the number of passes in the wet abrasion test are presented in Table 7 below.

TABLE 7

| | | Polish 3 | Polish 4 | Polish 5 | Polish 6 |
|---|---|---|---|---|---|
| 0 | pass | 100 | 100 | 100 | 100 |
| 2000 | passes | 100 | 99.7 | 100 | 100 |
| 4000 | passes | 99.5 | 99.1 | 99.6 | 99.4 |
| 6000 | passes | 99.3 | 98.5 | 99.5 | 99.4 |
| 8000 | passes | 99.1 | 98.1 | 99.2 | 99.0 |
| 10000 | passes | 98.8 | 97.7 | 98.9 | 98.7 |

TABLE 8

| Polish | Make-up time | Make-up removal time |
|---|---|---|
| Polish 3 | 5 minutes | 5 minutes with nail polish remover |
| | | based on ethyl acetate |
| Polish 4 | 5 minutes | 5 minutes with nail polish remover |
| | | based on ethyl acetate |
| Polish 5 | 5 minutes | 7 minutes with nail polish remover |
| | | based on ethyl acetate |
| Polish 6 | 5 minutes | 6 minutes with nail polish remover |
| | | based on ethyl acetate |

1. A nail polish composition comprising:
at least a film-forming agent, a plasticizer and a solvent, wherein said composition also comprises one or more oligomer(s) of molecular weight above 1500 g/mol and/or whose glass transition temperature Tg is above 25° C., said oligomer possessing acrylic or methacrylic double bonds available for reaction with radicals, but does not contain a crosslinking agent of the oligomer, such as a photoinitiator.

2. The nail polish composition as claimed in claim 1, wherein the molecular weight of the oligomer is greater than or equal to 2000 g/mol, preferably greater than or equal to 3000 g/mol.

3. The nail polish composition as claimed in claim 1, wherein the oligomer has a glass transition temperature Tg above 30° C., preferably above 35° C., more preferably above 40° C.

4. The nail polish composition as claimed in claim 1, wherein the oligomer has a Persoz hardness of at least 60 s, preferably at least 80 s, more preferably at least 100 s, when it is applied on a glass plate at a thickness of 100 μm when wet, and then dried for 24 h at 25° C. away from the light.

5. The nail polish composition as claimed in claim 1, wherein the oligomer is selected from: a (meth)acrylated poly(meth)acrylate, a (meth)acrylated polyester, a (meth)acrylated polyether, a (meth)acrylated urethane, a (meth)acrylated polyurethane or a (meth)acrylated epoxy, a (meth)acrylated silane-modified poly(meth)acrylate or a mixture thereof.

6. The nail polish composition as claimed in claim 1, wherein said composition contains a concentration of said oligomer between 0.1 and 30 wt %, preferably between 0.5 and 20 wt %, more preferably between 1 and 15 wt % of the total weight of the nail polish composition.

7. A method for applying polish on a nail comprising the following successive steps:
i) applying a first composition A as claimed in claim 1 on the nail in the form of at least one film FA, composition A preferably containing a colored pigment and not containing a photoinitiator; followed by drying of said film FA;
ii) applying, on the surface of the film FA formed by composition A, a second composition B, called a top coat, as claimed in any one of claims 1 to 6 and further comprising a photoinitiator, in the form of a film FB; said composition B containing a photoinitiator;
iii) initiating the crosslinking of composition B in visible light, the crosslinking of the oligomers of composition B causing crosslinking of the oligomers present on the surface of film FA, inducing a strong bond between films FA and FB.

8. A nail polish kit according to claim 1, wherein comprises:
a container CA, of the bottle type, containing a first nail polish composition A,
a container CB, of the bottle type, containing a second nail polish composition B and further comprising a photoinitiator,
instructions for use.

9. The kit as claimed in claim 8, wherein the photoinitiator of composition B is of the radical type capable of initiating crosslinking of the oligomer or oligomers (at least) in daylight.

10. The kit as claimed in claim 8, wherein the concentration of photoinitiator is between 0.1 and 10 wt %, preferably between 0.5 and 5 wt % of the total weight of the nail polish composition.

11. The kit as claimed in claim 8, wherein container CA has walls at least partially transparent so that the color of the colored polish of the first composition A is visible, when the latter is stored in said container CA.

12. The kit as claimed in claim 8, wherein container CB has walls that are opaque to visible light and to UV, and able to prevent crosslinking of the polish of the second composition B containing the photoinitiator, when the latter is stored in said container CB.

13. The kit as claimed in claim 8, wherein said kit comprises a container containing a nail polish remover suitable for removing the layers of polish A and B from the nail.
